(19) United States
(12) Patent Application Publication
(10) Pub. No.: US 2011/0104068 A1
(43) Pub. Date: May 5, 2011

(54) Echogenic Coatings and Their Use in Medical Devices

(76) Inventors: Harold Elbridge Garey, JR., Methuen, MA (US); Andrew Martin Reed, Boca Raton, FL (US)

(21) Appl. No.: 12/938,925

(22) Filed: Nov. 3, 2010

Related U.S. Application Data
(60) Provisional application No. 61/257,534, filed on Nov. 3, 2009.

(51) Int. Cl. A61K 49/22; A61P 43/00

(52) U.S. Cl. 424/9.5

(57) ABSTRACT

The present teachings relate to echogenic coatings and medical devices coated with an echogenic coating. The echogenic coating can be prepared from a composition comprising a polymer and an effervescent agent in one or more anhydrous solvents.

### CROSS REFERENCE TO RELATED APPLICATIONS

This application claims priority to and the benefit of U.S. Provisional Patent Application Ser. No. 61/257,534, filed on Nov. 3, 2009, the disclosure of which is incorporated by reference herein in its entirety.

### FIELD

The present teachings relate to echogenic coatings and their use.

### BACKGROUND

Ultrasonic imaging in the medical field is widely used for a variety of applications. In addition to imaging physiological structures and tissue such as organs, tumors, vessels, and the like, it is often desirable for a physician or technician to have an image of a medical device which has been inserted into the tissue or passageway of a patient.

However, most medical devices have an acoustic impedance similar to that of the tissue into which the device is inserted. Consequently, visibility of the device can be poor and accurate placement can become extremely difficult.

Gases being highly compressible have much lower acoustic impedance than liquids and solids. Accordingly, gas bubbles have a high degree of echogenicity (or ultrasonic visibility) compared to organ tissues because of their large impedance difference. While gas-filled microbubbles can be used as contrast agents, for example, by intravenous administration to systemic circulation, their practical use is limited by their short circulation residence times.

Accordingly, there remains a need in the art to improve the echogenicity of medical devices.

### SUMMARY

In light of the foregoing, the present teachings provide echogenic coatings that can be deposited on the surface of various medical devices to confer improved echogenicity. More specifically, the present coatings include a polymer matrix with effervescent agents dispersed therein that can react to generate microbubbles upon contact with a fluid (e.g., blood or water). The polymer used to form the matrix preferably is hydrophilic and has a small pore size. This helps ensure that while fluid can enter the matrix to initiate the gas-generating reaction, release of any microbubbles into the surrounding environment generally does not take place.

### DETAILED DESCRIPTION

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components or can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

Echogenic coatings of the present teachings can be prepared by providing a composition including a biocompatible polymer and an effervescent agent in an anhydrous solvent, depositing the composition on a substrate, and drying or curing the deposited composition.

The polymers used to form the polymer matrix preferably are biocompatible and highly hydrophilic, and have good tensile strength and adhesion to a wide array of metallic and polymeric substrates. Suitable polymers include those that have been used as hydrophilic polymeric coatings for medical devices such as poly-N-vinylpyrrolidone (PVP), polyethylene oxide (PEO), polyethylene glycol (PEG), polyvinylalcohol (PVA), polymethylmethacrylate (PMMA), polyurethane (PU), and copolymers thereof. Mixtures and blends of these polymers also can be used. The resulting polymer matrix has small pore size such that gas bubbles once formed are trapped within the matrix.

The effervescent agent can be various compounds that evolve gas. In various embodiments, the effervescent agent generates a gas by means of a chemical reaction upon exposure to water or other fluids. For example, the effervescent agent can include an agent capable of releasing carbon dioxide (e.g., a carbonate source) and an agent which induces the release of carbon dioxide (e.g., an acid source), typically in a 1:1 ratio. Suitable agents capable of releasing carbon dioxide include alkali metal carbonates and alkali metal bicarbonates such as carbonates or bicarbonates of sodium, potassium, or calcium. Suitable agents capable of inducing the release of carbon dioxide include various edible organic acids or their acidic salts. Suitable edible organic acids include tartaric acid, malic acid, fumaric acid, adipic acid, succinic acid, ascorbic acid, maleic acid, or citric acid. Suitable acidic salts include salts of polybasic acids which are present in solid form and in which at least one acid function is present such as potassiumm dihydrogen or disodium hydrogen phosphate or the corresponding citrates. To prevent premature evolution of carbon dioxide, these agents preferably are provided as a mixture in powder form and are kept in a generally anhydrous state until it is desirable to activate them with water or other fluids. In a preferred embodiment, the effervescent agent can include sodium bicarbonate and citric acid powder. To further prevent the effervescent agent from premature reaction, a surface-modified sodium bicarbonate powder can be used. For example, the surface of sodium bicarbonate particles can be converted to sodium carbonate.

The effervescent agent and the polymer can be mixed together in one or more organic solvents to provide a coating composition. Suitable solvents that can be used include tetrahydrofuran, acetone, methylethylketone, dimethylformamide, dimethyacetamide, ethylene carbonate, propylene carbonate, diglyme, N-methylpyrrolidone, ethyl acetate, ethylene and propylene glycol diacetates, alkyl ethers of ethylene and propylene glycol monoacetates, toluene, xylene and sterically hindered alcohols such as t-butanol and diacetone alcohol. In preferred embodiments, the organic solvent or solvent mixture is water-miscible. For example, tetrahydrofuran or a mixture comprising tetrahydrofuran and dimethyacetamide (e.g., at a ratio of 95:5) can be used. The total solid loading can be between about 5 wt. % and about 30 wt. %, where the loading of the effervescent agent is between about 0.01 wt. % and about 5 wt. % of that of the polymer.

To improve the echogenicity of a medical device, at least a portion of the surface of the medical device can be coated with the present coating composition. Various coating techniques such as spin coating, drop-casting, zone casting, dip coating, blade coating, and spraying can be used, depending on the shape of the medical device. For example, the medical device can be an elongated member such as a catheter, a guidewire, or a needle, or a planar or spherical member such as an implant or a balloon. The thickness of the coating should be sufficient to entrap gas bubbles having a diameter between about 1 μm and about 50 μm. Accordingly, typical thickness of the coating can range from about 0.01 mm to about 0.1 mm. The thickness achieved by one application of the coating composition will depend on the viscosity of the coating composition, the coating method, as well as the speed and the temperature at which the coating is applied. In some embodiments, multiple applications of the coating may be needed to build up the required thickness. The coating is then allowed to dry. Depending on the polymer, curing (e.g., via thermal or photo crosslinking) can be necessary to provide the coating with the requisite mechanical strength.

The coating can be activated prior to use or during use. For example, in certain applications, the coating can be exposed to water before the medical device is inserted into a patient's body. In other applications, bubbles are generated in vivo upon contact of the coating with blood. Because of the hydrophilicity of the polymer, water molecules in the blood plasma can permeate the polymer matrix efficiently to dissolve the effervescent agent, which leads to the generation of a large amount of gas bubbles. Due to the small pore size of the polymer matrix, substantially all of the gas bubbles will stay entrapped within the coating instead of escaping into the bloodstream. Once activated, the medical device coated with the present echogenic coating can be viewed by an ultrasound imaging apparatus with enhanced visibility.

In alternative embodiments, instead of mixing the polymer and the effervescent agent in a solvent to prepare a coating composition, the effervescent agent can be dispersed in a polymer melt and/or added as an additive during preparation of a polymer to provide an echogenic polymeric material. In embodiments where the effervescent agent is incorporated into a polymer melt or where the polymerization reaction is conducted neat (i.e., without a solvent), the polymer melt or the polymerization mixture should have a sufficiently low viscosity to allow effective dispersion of the effervescent agent. The resulting echogenic polymeric material can then be extruded or molded into a medical device or components thereof. Similar to previous embodiments, medical devices incorporating the echogenic polymeric material can be activated prior to use or during use by immersion in water or other fluids.

The present teachings can be embodied in other specific forms without departing from the spirit or essential characteristics thereof. The foregoing embodiments are therefore to be considered in all respects illustrative rather than limiting on the present teachings described herein. The scope of the present teachings is thus indicated by the appended claims rather than by the foregoing description, and all changes that come within the meaning and range of equivalency of the claims are intended to be embraced therein.

What is claimed is:

1. A medical device comprising an echogenic coating, wherein the echogenic coating comprises an effervescent agent dispersed within a polymer matrix.

2. The medical device of claim 1, wherein the polymer matrix comprises polyurethane.

3. The medical device of claim 1, wherein the effervescent agent comprises an agent capable of releasing carbon dioxide and an agent capable of inducing the release of carbon dioxide.

4. The medical device of claim 3, wherein the agent capable of releasing carbon dioxide comprises one or more of sodium carbonate, sodium bicarbonate, potassium carbonate, and calcium carbonate.

5. The medical device of claim 3, wherein the agent capable of inducing the release of carbon dioxide comprises one or more of tartaric acid, malic acid, fumaric acid, adipic acid, succinic acid, ascorbic acid, maleic acid, and citric acid.

6. A composition for forming an echogenic coating, wherein the composition comprises an effervescent agent and a polymer dissolved in one or more anhydrous solvents.

7. The composition of claim 6, wherein the polymer is a polyurethane.

8. The composition of claim 6, wherein the effervescent agent comprises an agent capable of releasing carbon dioxide and an agent capable of inducing the release of carbon dioxide.

9. The composition of claim 8, wherein the agent capable of releasing carbon dioxide comprises one or more of sodium carbonate, sodium bicarbonate, potassium carbonate, and calcium carbonate.

10. The composition of claim 8, wherein the agent capable of inducing the release of carbon dioxide comprises one or more of tartaric acid, malic acid, fumaric acid, adipic acid, succinic acid, ascorbic acid, maleic acid, and citric acid.

11. The composition of claim 6, comprising a solid loading between about 5 wt. % and about 30 wt. %.

12. The composition of claim 11, wherein the loading of the effervescent agent is between about 0.01 wt. % and about 5 wt. % of that of the polymer.
